(19) United States
(12) Patent Application Publication
(10) Pub. No.: US 2019/0292608 A1
(43) Pub. Date: Sep. 26, 2019

(54) Methods To Quantify Bioburden In Substances

(71) Applicant: Buckman Laboratories International, Inc., Memphis, TN (US)

(72) Inventors: Mark L. Reed, Cordova, TN (US); Justin Hutcherson, Memphis, TN (US)

(73) Assignee: Buckman Laboratories International, Inc., Memphis, TN (US)

(21) Appl. No.: 16/285,251

(22) Filed: Feb. 26, 2019

Related U.S. Application Data
(60) Provisional application No. 62/670,056, filed on May 11, 2018.
(60) Provisional application No. 62/648,013, filed on Mar. 26, 2018.

(51) Int. Cl. C12Q 1/6888

(52) U.S. Cl. CPC C12Q 1/6888

(57) ABSTRACT

Methods to quantify bioburden present in industrial substances are provided that utilize a polymerase chain reaction (PCR) and detection of amplification signals over multiple PCR thermal cycles. The PCR targets a segment of DNA found in one or more biofouling agents that are often found in industrial substances. A sample taken from the substance can be used directly or first filtered, purified, lysed, diluted, or subjected to a combination of such pretreatments. The substance is used in, or is tested for preparedness to be used in, industrial applications and sites, such as non-pharmaceutical applications, non-medical applications, papermaking facilities, leather-treating facilities, and the like. Methods are provided to control or treat substances or systems utilizing such substances, or to control or treat surfaces intended to come into contact with the substances.

This application claims the benefit under 35 U.S.C. § 119(e) of prior U.S. Provisional Patent Application Nos. 62/648,013, filed Mar. 26, 2018, and 62/670,056, filed May 11, 2018, which are incorporated in their entireties by reference herein.

### BACKGROUND OF THE INVENTION

The present invention relates to bioburden in industrial substances or in industrial applications. In more detail, the present invention relates to methods to quantify or understand or determine bioburden in substances present or used in industrial applications, such as, but not limited to, papermaking, pulp production, leather processing, oil and gas recovery or production, fermentation processes, water treatment, and/or sewage treatment.

Bioburden quantification can be expressed in colony forming units (CFU). Bioburden is also associated with biofouling, for example, wherein microbes collect on the surface of a device or inside of fan cooled equipment. This can increase the risk of human infections. Bioburden is normally defined as the number of bacteria living on a surface that has not been sterilized. Bioburden testing for medical devices made or used in the USA is governed by Title 21 of the Code of Federal Regulations and worldwide by ISO 11737.

The aim of bioburden testing is to measure the total number of viable micro-organisms (total microbial count). In industrial manufacturing and production and treatment facilities, understanding the bioburden is important so that appropriate steps can be considered and/or taken to avoid or control biofouling of the products being produced and/or avoid biofouling of the machinery or systems or raw materials or processing materials being used at the manufacturing and production and treatment facilities.

Currently, in industrial manufacturing and production and treatment facilities, bioburden is, for the most part, studied or estimated using petri films or an ATP test. The ATP test is a process of rapidly measuring actively growing microorganisms through detection of adenosine triphosphate. The ATP test, while fast, provides results that are considered less sensitive and/or provides results with increased variability and/or provides results with ‘noise’. Put another way, the ATP test may provide a general understanding of bioburden but not a very accurate understanding that can be used to quantify the bioburden with respect to the details of the bioburden, so that, for instance, treatment of the bioburden can be sufficient and without wasting treatment chemicals by overdosing.

The other method commonly used in industrial applications is the use of conventional plating methods where the agar is prepared or using petrifilm plates that provide an all-in-one plating system. While the petrifilm plates and method are used because of their cost-effectiveness, simplicity, convenience, and ease of use, the method suffers from taking time in obtaining results, sometimes 24 hours to 48 hours, and the method further suffers from not being specific to the exact organism being grown or multiplied on the petrifilm The plating method is limited to a defined media that typically does not resemble an actual sample and thus limits the organisms that can grow successfully.

Accordingly, a need exists for methods that can address the problems with current methods to understand bioburden. Further, there is a need to provide methods that can quantify bioburden more accurately and/or in a rapid manner and/or provide bioburden details with respect to specific species of organisms, which all leads to a more effective ability to understand and thus treat or control the bioburden.

### SUMMARY OF THE PRESENT INVENTION

A feature of the present invention is to provide rapid method for enumeration of biofouling agents in a range of industrial processes. Biofouling agents that can be tested for and enumerated according to the present invention include, but are not limited to, bacteria, algae, fungi, and/or yeast. Enumerating biofouling agents such as bacteria can be important in determining whether treatment of a liquid, pulp, water, brine, tanning solution, suspension, dispersion, emulsion, sludge, or other substance, is needed or recommended, and how much treatment agent should be used. For example, by enumerating or quantifying colonies of bacteria in an industrial sample such as a papermaking pulp, an effective yet minimum amount of biocide can be calculated, computed, or otherwise determined that can be dosed into the papermaking pulp for the purpose of controlling and/or killing the bacteria. The use of excess biocide can be avoided, and a cost-effective treatment can result with minimum biocide contamination and consumption.

The detection methods and treatments according to the present teachings can be used in industries including, but not limited to, papermaking pulp production, leather processing, oil and gas recovery or production, fermentation processes, cooling water towers, water treatment, and/or sewage treatment. According to the present invention, many different biofouling agents can be tested for and enumerated, quickly and simultaneously. The enumeration can be limited to a specific biofouling agent, commonly or often found in a particular industrial substance, or to a set or group of biofouling agents. For example, analysis of water from a cooling water tower can include the enumeration of bacteria and algae, analysis of a leather-treating brine or wash can include the enumeration of bacteria and fungi, and analysis of a paper-making pulp can include the enumeration of bacteria, algae, and fungi. The analysis can be tailored, for example, to the specific industrial substance, the environment, previous test results, other historical data, or a combination thereof.

Methods according to the present teachings can be used to quantify bioburden present in a substance. The method can involve obtaining a sample of the substance, optionally filtering the sample, optionally diluting the sample, and conducting a polymerase chain reaction (PCR) with the sample or a portion thereof. The PCR can be carried out in the presence of a primer pair configured to amplify a sequence or segment of the DNA of at least one organism suspected of causing a bioburden in the substance. For samples where known types or species of bacteria or other biofouling organisms are likely or known to be present, specific primer pairs can be used to target the types or species. For samples in which it is unknown what types or species of organism(s) may or may not be present and likely to cause bioburden, a variety of primer pairs can be used, or a universal primer pair can be used to amplify a sequence or segment of DNA of the at least one organism.

Primer pairs can be used that target a specific organism or to target a specific gene. For example, a primer pair can be used to enumerate the 16S gene, found in practically all known bacteria. Thus, even without targeting a specific type of bacteria, biofouling attributable to bacteria, in general, can be analyzed. Similarly, a primer pair that targets the 18S gene, found in practically all known eukaryotes, can be used to enumerate algae even without targeting a specific type of algae.

The method can involve amplifying a sequence or probe segment of DNA of the at least one organism over many thermal cycles, as is well known in the art. The rate of amplification with respect to the number of thermal cycles can be detected, for example, by well-known fluorescent detection methods, and the fluorescent emission can be graphed (or otherwise plotted or analyzed) with respect to cycle number to generate a fluorescent curve or signal that can be compared to or correlated with a standard, standard curve, look-up table, or other predetermined values to determine an estimated amount or concentration of the biofouling organism in the substance.

Fluorescent detection can be used to measure an amount of PCR product (amplicons), as is well-known in the art. Intercalating dyes, for example, SYBR® green (ThermoFisher Scientific, Waltham, Mass.), and EvaGreen® (Biotium, Fremont, Calif.) can be used. Fluorescent probes, including, for example, FRET dyes having reporter and quenchers dyes, can be used and are well-known in the art. Specific probes configured to anneal to known DNA sequences or segments, of known biofouling agents, can be used and can be made with specific reporter dyes, for example, to optimize detection using a particular detection system. Different reporter dyes can be used on different probes so that more than one biofouling agent can be detected in, and enumerated from, a single sample, for example, by using wavelength filters for excitation radiation, emission radiation, or both.

Another feature of the present invention is to provide a quick, efficient, and inexpensive method of determining an estimated amount or concentration of a biofouling organism in a substance, and that can be carried out at an industrial site (on-site and portable), without the need to send a sample to a laboratory and await results. Such a feature is, in part, enabled by recent developments in polymerase chain reaction (PCR) methods and machines, which have provided low cost options for portably carrying out PCR on-site and within three hours or less, for example, within two hours or less or within one hour or less.

A further feature of the present invention is a method to generate standards for comparing or correlating a quantitative PCR (qPCR) result to surmise or calculate an estimated amount or concentration of a biofouling organism in a substance. Known concentrations of a species of a biofouling organism can be prepared in known industrial substances and can be subject to PCR (e.g. qPCR) under a specific set of conditions including, for example, a specific polymerase enzyme, in the presence of a specific primer pair, a specific set of reagents, a specific detectable dye, and/or at a specific thermocycling temperature profile, to generate threshold cycle values that differ depending upon the initial concentration of the species in the sample. The quantification cycle (Cq) or threshold cycle (Ct) detected for the differing initial concentrations can be determined by fluorescence detection as is well-known in the art of thermal cycling and polymerase chain reaction assays. The generated standard graphs, standard slopes, standard curves, or other standard values can be printed, displayed, stored in a memory, listed in a look-up table, or otherwise made available for comparison to a Cq value, a Cq graph, a Cq slope, a Cq curve, a Ct value, a Ct graph, a Ct slope, or a Ct curve generated by an unknown sample. The standard or set of standards can be compared to results obtained from subjecting a sample from an industrial site, having an unknown concentration of the same biofouling agents, to the same conditions.

Additional features and advantages of the present invention will be set-forth in part in the description that follows, and in part will be apparent from the description, or may be learned by practice of the present invention. The objectives and other advantages of the present invention will be realized and attained by means of the elements and combinations particularly pointed out in the description and appended claims.

It is to be understood that both the foregoing general description and the following detailed description are exemplary and explanatory only and intended to provide a further explanation of the present invention, as claimed.

### BRIEF DESCRIPTION OF DRAWINGS

FIGS. 1A and 1B are parts one and two of a table showing primers that can be used as primer pairs for replicating DNA sequences of known biofouling organisms found in industrial substances. Each figure lists the corresponding SEQ ID NO: for the sequence listed in the figures.

FIG. 2 is a table showing standard values of Cq of industrial samples having known biofouling agent cell concentrations per microliter (μL) and showing that calculated concentrations based on Cq measurements show good correspondence to the known, actual, given concentrations. To be used as a measure of quantity, the Cq of a sample can either be related to the Cq of another sample (often named a calibrator), to determine relative quantification, or be related to the Cq of a set of known copy number standards, to determine absolute quantification.

FIG. 3 is a graph showing a standard curve or line correlating the quantification cycle values (Cq) of the various serial dilutions of cells shown in FIG. 2. The cells are of a known biofouling organism. The data and/or graph can be used as a standard or set of standards, or to prepare a standard or set of standards, for example, to compare on-site test results of a sample from an industrial substance. Standards based on Cq or the cycle threshold value (Ct) can be used as, or used to generate, standards.

FIG. 4 is a table showing leather brine samples and wash samples that include biofouling agents that, when subject to PCR, generate Cq values indicating the concentrations of agents shown in the table. Each sample was diluted by a factor of 1000 before running the PCR used to generate the Cq values.

### DETAILED DESCRIPTION OF THE PRESENT INVENTION

The present teachings provide a method to quantify or enumerate bioburden present in industrial substances, which utilizes polymerase chain reaction (PCR) and detection of the rate of change of the number of PCR amplicons over multiple PCR thermal cycles. The PCR can be designed to target one or more segments of DNA found in one or more biofouling agents that are often found in such industrial substances. As an example, Enterobacter bacteria can often be found in, and can biofoul papermaking pulp. The present teachings encompass methods for targeting one or more signature DNA segments of Enterobacter for the purpose of determining the presence and concentration of the bacteria in the pulp. One or more appropriate biocides can then be added to or mixed with the pulp, to control, reduce, or eliminate the bioburden that would otherwise result from the presence of the bacteria. A correct dosage of biocide can thus be used, based on the calculated or computed concentration of the bacteria in the pulp. The use of excess biocide and excess costs associated therewith, can be avoided.

A sample can be taken from a substance and used directly or first filtered, purified, lysed, diluted, or subjected to a combination of such pretreatments. The sample size can be any suitable volume, for example, from 0.1 microliter (μl) to 10 milliliters (ml), from 1 μl to 5 ml, from 10 μl to 2 ml, from 100 μl to 1 ml, or about 1 ml. The sample can then be diluted. or used as-is. As an example, a 1 ml sample can be taken, then diluted by a factor of 10, a factor of 100, or a factor of 1000, and an aliquot of the diluted sample can then be used, such as a 1 μl aliquot of a 1 ml sample that has been diluted. The substance can be any of a wide variety of industrial substances used in or at industrial applications and sites, such as non-pharmaceutical applications, non-medical applications, papermaking facilities, alcohol production facilities, fermentation facilities, leather-treating facilities, and the like. The methods of the present invention can also be carried out additionally, or instead, at a laboratory or other remote location. The method can be used to control or treat substances or systems utilizing the substances, or to control or treat surfaces intended to come in contact with the substances.

The present invention relates to a method for rapidly enumerating bacteria in a range of industrial processes. Enumerating bacteria or microorganisms can be important in determining whether treatment of a liquid, pulp, water, brine, tanning solution, suspension, dispersion, emulsion, mixture, sludge, or other substance is needed or recommended, and how much treatment agent should be used. For example, by enumerating or quantifying colonies of bacteria (and/or other microorganisms) in an industrial sample such as a papermaking pulp, an effective yet minimum amount of biocide (or microbiocide) can be determined that can be dosed into the papermaking pulp for the purpose of controlling or reducing the microorganism count. The use of excess biocide can be avoided and a cost-effective treatment results with minimum biocide contamination and consumption.

The detection methods and treatments according to the present teachings can be used in industries including, but not limited to, papermaking pulp production, leather processing, oil and gas recovery or production, fermentation processes, water treatment, cooling water systems, and/or sewage treatment.

Methods according to the present teachings can be used to quantify bioburden present in a substance. The method can involve obtaining a sample of the substance, optionally filtering the sample, optionally diluting said sample, and conducting a polymerase chain reaction (PCR) with the sample, or a portion thereof, in the presence of a primer pair for amplifying and detection reagents for detecting a sequence or segment of DNA of at least one organism suspected of causing a bioburden in the substance. For samples in which known types of bacteria or other fouling organisms are likely or known to be present, specific primer pairs can be used to target the species of bacteria or other fouling organisms. For samples in which it is unknown what species may or may not be present and likely to cause bioburden, a variety of primer pairs can be used, a universal primer pair can be used, or a primer pair directed to a common gene can be used to amplify a sequence or segment of DNA of the at least one organism or gene.

The method can involve amplifying a sequence or segment of DNA of the at least one organism over many thermal cycles, as is well-known in the art. The rate of amplification with respect to the number of thermal cycles can be detected, for example, by well-known fluorescent detection methods. The fluorescent emission can be graphed or otherwise plotted with respect to the cycle number, to generate a fluorescent curve or signal that can be compared to or correlated with a standard, set of standards, standard curve, look-up table, stored values, or other predetermined values, to determine an estimated amount or concentration of the biofouling organism in the substance, i.e., to enumerate the biofouling agent.

According to present teachings, the method can comprise obtaining a sample of the substance, optionally filtering the sample, optionally diluting said sample, and conducting a polymerase chain reaction (PCR) with the sample. Amplification data resulting from amplification of the sequence or segment of DNA can then be measured, determined, or calculated and then correlated with one or more standards to determine an estimated amount or concentration of the organism in the substance. The amplification data can comprise a rate of amplification, a Cq value, a Ct value, fluorescence emission intensity data, or a combination thereof. The substance can be an industrial substance from, or supplied to, a papermaking plant, a pulp making plant, a leather making or processing plant, a fermentation facility, an oil and gas recovery site or production facility, a water treatment plant, a water-cooling facility, or a sewage treatment plant. If necessary, the sample can be cleaned, purified, isolated, or otherwise treated to remove components that might otherwise interfere with the PCR. As an example, a sample can be lysed and filtered before being subjected to PCR.

The sample can comprise cells, endospores, live organisms, or viable cells from an organism, or a combination thereof. If for example, the sample comprises endospores and viable cells, at least two primer pairs can be used, and each primer pair can target and amplify a respective segment of DNA of at least one organism that causes bioburden in the industrial substance. The sample can comprise endospores and viable cells, and the method can comprise isolating the endospores from the sample prior to conducting the PCR on the endospore fraction. The method can comprise isolating viable cells from the sample prior to conducting the PCR on the viable cell fraction. The method can involve preparing the sample. If the sample comprises viable cells of a biofouling organism, the preparation can include lysing the viable cells.

The industrial substance can primarily comprise a liquid, pulp, water, brine, tanning liquor, solution, dispersion, suspension, emulsion, mixture, solid, sludge, or a combination thereof. The substance can comprise a liquid and a solid, and the method can comprise substantially removing the solid before conducting the PCR. The method can comprise treating the substance at a location having the substance, for example, directly at the location from which the sample was taken (e.g., on-site analysis and treatment). The treatment can involve administering at least one biocide or microbiocide, based on the estimated amount or concentration of the biofouling organism detected in the substance, that is, based on the calculated or computed bioburden. The treatment can involve treating the substance at a processing facility retaining, containing, or configured to process, the substance. Treatment with at least one biocide or microbiocide can be administered at a dosage calculated based on the estimated amount or concentration of the organism(s) in, or bioburden of, the substance.

The method can further comprise diluting the sample to form at least a first sample portion and a second sample portion. The sample can be diluted, for example, serially diluted. Three, four, or more serial dilutions can be made and tested. Sometimes, a one degree of dilution can lead to better, more clear results, with a smaller standard deviation. With differently diluted samples, conducting the PCR can comprise conducting PCR on a first sample portion utilizing a primer pair to target and amplify a segment of DNA of at least one organism that is known to cause a bioburden in said substance, and conducting PCR with a second sample portion utilizing the primer pair or a different primer pair. The rate of amplification, Cq or Ct of the segment of DNA, determined from the PCR carried out on the first sample portion, can be correlated with a standard or set of standards to determine a first estimated amount or concentration of an organism in the substance. The rate of amplification, Cq, or Ct of the segment of DNA, determined from PCR carried out on the second sample portion, can be correlated with the same standard or set of standards to determine a second estimated amount or concentration of the organism in the substance. Moreover, the first estimated amount or concentration can be compared with the second estimated amount or concentration and an average amount or concentration can be determined, along with a standard deviation. From the results, a dosage of a biocide or microbiocide can be administered, to the substance, wherein the dosage is calculated based on the average amount or concentration. When higher concentrations of biofouling agent are determined to be present, higher dosages of biocide can be used to treat the substance. As an example, the dosage of biocide can be doubled with each Cq reduction of five, so that, for an industrial substance such as a papermaking pulp, that exhibits a Cq of 15, twice as much biocide can be administered relative to the amount of biocide administered for the otherwise same substance but which exhibits a Cq of 20.

The primer pairs can be used together with a probe having at least one conjugated or bound fluorescent dye. The annealing of the primer pair and probe to the segment of DNA, followed by extension, can result in an unquenched fluorescent dye, as is well-known in the art. An excitation wavelength can be used to excite the unquenched dye and the fluorescence emission spectrum and intensity can be used to plot a Cq or Ct graph over the number of thermal cycles. The method excitation wavelength can be directed toward the sample, which excites the unquenched fluorescent dye, during each thermal cycle of the PCR. The intensity of fluorescence emission can be detected, measured, or sensed during each thermal cycle of the PCR. The rate of amplification of the segment of DNA, or a Cq or Ct, can be determined by graphing a curve of the intensity of fluorescence emission, per thermal cycle. If a threshold cycle (Ct) is determined, it can be correlated to the Ct values of a known standard or set of standards having known concentrations of the at least one organism. If a quantification cycle (Cq) is determined, it can be correlated to the Cq values of a known standard or set of standards having known concentrations of the at least one organism. The method can further comprise generating and detecting a signal that (1) is indicative of the presence of a segment of DNA, and (2) intensifies with an increasing concentration of amplicons of the segment of DNA, resulting from the PCR.

The primer pair can comprise a universal primer pair including a forward primer and a reverse primer designed to amplify segments of DNA of more than one different organism that is known to cause bioburden in the substance. The primer pair can be designed to amplify one or more segments of DNA from bacteria. The primer pair can comprise a primer pair designed to amplify one or more segments of DNA from one or more fungi. The primer can comprise a primer designed to amplify one or more segments of the 16S gene or the 18S gene.

The method can be used to detect endospores. An endospore is a dormant, tough, and non-reproductive structure produced by certain bacteria most commonly from the Firmicute phylum. It is a stripped-down, dormant form to which the bacterium can reduce itself. Endospore formation is usually triggered by a lack of nutrients, and usually occurs in Gram-positive bacteria. Endospores enable bacteria to lie dormant for extended periods, even centuries. When the environment becomes more favorable, the endospore can reactivate itself to the vegetative state. By being able to detect endospores, forms of bacteria can be detected when the bacteria themselves cannot be.

Some classes of bacteria can turn into exospores, also known as microbial cysts, instead of endospores. Exospores and endospores are two kinds of “hibernating” or dormant stages seen in some classes of microorganisms. The methods can detect and quantify the bacterium's DNA present in an endospore or in an exospore.

Another feature of the present invention is to provide a quick, efficient, and inexpensive method of determining an estimated amount or concentration of the biofouling organism in the substance, and that can be carried out at an industrial site without the need to send a sample to a laboratory and await results. The present invention permits on-site analysis (at the site of the substance being analyzed) and permits a quantifying within 3 hours or less, two hours or less, one hour or less, from 20 minutes to 3 hours, from 30 minutes to 2 hours, or from 30 minutes to 1 hour.

The present invention further permits the ability or option to avoid DNA extraction or purification which can be time consuming.

The present invention uses polymerase chain reaction (PCR) methods and machines, which have provided low cost options for portably carrying out PCR on-site and within three hours or less, for example, within two hours or within one hour. As an example, a magnetic induction thermal cycler can be used. An exemplary machine that can provide portability, that ability to perform PCR on a sample at an industrial site, and very rapid cycling times, for example, 30 thermal cycles in three hours or less, two hours or less, or one hour less, is the MIC magnetic induction cycler manufactured by Bio Molecular Systems, and available from Bioline USA Inc or Taunton Mass. Any suitable thermocycling PCR machine can be used and devices other than magnetic induction cyclers can be used. Thermocyclers having liquid heated and cooled thermocycling blocks can be used, thermocyclers having Peltier heating elements can be used, and the like. The same machine or same model of machine used to generate a standard or set of standards can be used to test an industrial substance by comparison to the standard or set of standards. Standards or sets of standards can be generated in a laboratory using well-known PCR instruments that take a relatively long time to generate results. On-site testing and comparison to a standard or set of standards can use a portable, relatively faster machine, for example, the MIC magnetic induction cycler described above.

A further feature of the present invention is a method to generate standards for comparing or correlating quantitative PCR (qPCR) result to surmise or calculate an estimated amount or concentration of a biofouling organism in a substance. Known concentrations of a species of a biofouling organism can be prepared in known industrial substances and can be subjected to PCR under a specific set of conditions including, for example, a specific polymerase enzyme, in the presence of a specific primer pair, in the presence of a specific set of reagents, in the presence of a specific fluorescent dye, and at a specific thermocycling temperature profile, to generate threshold cycle values that differ depending upon the initial concentration of the species in the sample. The quantification cycle (Cq) or threshold cycle (Ct) detected for the differing initial concentrations can be determined by fluorescence detection as is well-known in the art of thermal cycling and polymerase chain reaction assays. The generated standard graphs, standard slopes, standard curves, or other standard values can be printed, displayed, stored in a memory, or otherwise made available for comparison to a Cq value, a Cq graph, a Cq slope, a Cq curve, a Ct value, a Ct graph, a Ct slope, or a Ct curve generated by subjecting a sample having an unknown concentration of the same species, under the same conditions, and tested on-site.

For generating standards, a method of calibrating the temperature to be used for thermal cycling can be implemented. The calibrating can involve cycling temperatures of a set of wells, using spectrally distinguishable species, and measuring the signal from each well during different temperature cycles, as described, for example, in U.S. Pat. No. 7,875,425 B2 to Gunstream et al., which is incorporated herein in its entirety by reference.

The method can involve using the slope of a standard curve to estimate PCR amplification efficiency. A qPCR standard curve can be graphically represented as a semi-log regression line plot. If the starting template is RNA, a qPCR standard curve can be graphically represented as a semi-log regression line plot of Cq value vs. log of input cDNA. As described in Enke, R, qPCR Primer Efficiency Standard Curve Analysis, CSHL DNALC RNA-Seq for the Next Generation Working Group (2016), http://www.rnaseqforthxeneration.org/profiles/raymond-enke.html#teaching (the Enke article), a slope of −3.32 indicates a PCR reaction with 100% efficiency. More negative slopes indicate reactions that are less than 100% efficient. More positive slopes indicate sample quality or pipetting problems. A 100% efficient reaction will yield a 10-fold increase in PCR amplicon every 3.32 cycles during the exponential phase of amplification (log 2 10=3.3219). Percent PCR efficiency can then be calculated from the slope:

PCR efficiency=[10(−1/m)]−1

where m=slope; efficiency of 100=1. The Enke article is hereby incorporated by reference herein, in its entirety. Melt curve analysis can also, or instead, be used to determine whether the amplification product resulting from PCR is a singular product or results from multiple different species or DNA segments, for example, as also described in the Enke article.

The method can involve detecting at least one target nucleic acid in a cell, for example, to detect a bioburden bacteria or microorganism(s) in a paper making pulp or other substances. The method can involve: (a) lysing the cell in a multifunctional lysis buffer to produce a cell lysate; and (b) detecting at least one target nucleic acid in the cell lysate using a quantitative nucleic acid detection assay. Exemplary quantitative nucleic acid detection assays that can be used include qPCR and other methods as described, for example, in U.S. Pat. No. 8,012,685 B2 to Shannon et al., which is incorporated herein in its entirety by reference.

To detect the amplification products, i.e., amplicons, intercalating dyes or detectable probes can be used. As is known in the art, fluorescent resonance energy transfer (FRET) dyes can be paired and attached to opposite ends of a DNA probe oligonucleotide sequence to form a detectable probe. The pair can include a reporter dye and a quencher dye. In a free-floating state, that is, before being annealed to a target sequence and then cleaved by a polymerase, fluorescent emissions from the reporter dye are quenched by the quencher dye that remains in close proximity to the reporter dye. Once the probe is annealed to a target, however, and during the extension phase of PCR, the exonuclease activity of the polymerase enzyme destroys the probe and physically separates the reporter dye from the quencher dye, resulting in a permanent increase in fluorescence. The fluorescence can be monitored, recorded, and charted in a way that enables measurement and/or computation of Ct and Cq values. The fluorescent reporter dye can be excited upon irradiation with an excitation wavelength to generate increased fluorescence that is detectable and indicative of the presence of the target DNA sequence. The more target molecules that are present, the more primers and probes that react and the more fluorescent reporter dye molecules that become unquenched. In samples having high starting concentrations of the target molecule or target DNA sequence, the fewer thermal cycles it takes to generate a maximum or plateaued fluorescent signal and the lower the number of the threshold cycle (Ct) and quantitation cycle (Cq).

Very tight temperature control and slightly varying temperatures of the thermal cycling cycle can be used to generate optimum results. Sometimes, varying an annealing temperature or denaturation temperature by just a single degree Celsius, or less, can improve amplification results and detectable signal intensity. Optimizing temperatures and other conditions during generation of a standard or set of standards can be helpful in determining the same temperatures and other conditions that should be used, on-site, to test an industrial sample. Apparatus, systems, and methods that can be used to optimize temperature profiles for thermal cycling include those described, for example, in U.S. Pat. No. 7,238,517 B2 to Atwood et al., which is incorporated herein in its entirety by reference.

A suitable kit can include reagents suitable for carrying out a plurality of single-plex quantitative or real-time amplification reactions. Such reagents can include a set of quantitative or real-time amplification primers, an oligonucleotide probe labeled with a labeling system suitable for monitoring the quantitative real-time amplification reaction, such as a FRET probe, a DNA polymerase at a concentration suitable for single-plex amplification, and mixtures of dNTPs suitable for template-dependent DNA synthesis.

The qPCR testing of a sample, and the qPCR sample processing used to generate standards, can be carried out using reagents and kits suitable for such amplifications. Such kits can include a plurality of amplification primer sets suitable for carrying out a single or multiplexed amplification. The primer sets can be individually packaged or packaged in a single container. The kit may optionally include one or more additional reagents for carrying out the amplification, such as a DNA polymerase enzyme, a reverse transcriptase enzyme, and mixtures of nucleoside triphosphates (“dNTPs”) suitable for extension of the primers via template-dependent DNA synthesis. The amount of optional polymerase included in the kit may be suitable for optimizing the efficiency of the amplification reaction. The various reagents may be packaged in combinations for maximum convenience and may be modeled after the combinations of reagents available commercially for carrying out conventional PCR and/or RT-PCR amplification reactions. Exemplary kits can include, and the methods can use, TAQMAN® Universal PCR Master Mix and/or a TAQMAN® Gold RT-PCR Kit available from Applied Biosystems, Foster City, Calif. The kit may further include other reagents, such as “tailed” primers as described, for example, in: Bengra et al., 2002, Clin, Chem. 48:2131-2140; Myakishev et al., 2001, Genome Res. 11:163-169; and U.S. Pat. No. 6,395,486), which are incorporated herein in their entireties by reference.

The methods can involve amplifying polynucleotide sequences of interest in a multiplex fashion to determine the presence and amount of more than one target or organism, at the same time. Methods, reagents, and kits useful for such purposes include those described, for example, in U.S. Pat. No. 8,323,897 B2 to Anderson et al., which is incorporated herein in its entirety by reference. One or more polynucleotides can be amplified, for example, by the polymerase chain reaction (“PCR”) or reverse-transcription polymerase chain reaction (“RT-PCR”), using a plurality of amplification primer pairs or sets, each of which is suitable or operative for amplifying a different polynucleotide sequence of interest, and using different detectable probes, one for each target. Probes can be selected that emit fluorescence at different wavelengths, so it can be discerned which of the multiple different target sequences are present and amplified. The multiplex amplification methods permit the simultaneous amplification and detection of a plurality of different sequences of interest in a single reaction vessel. The multiplex amplifications may be used in a variety of contexts to effectively increase the concentration or quantity of a sample available for quantitative analysis.

Both DNA and RNA target polynucleotides can be multiplex amplified using low primer concentrations. Specifically, the reverse transcription of RNA into cDNA via a reverse-transcription and subsequent multiplex amplification of the resultant cDNA with a DNA polymerase may be accomplished using primers at low concentrations (e.g., 45 nM for each primer). The amplification of both DNA and RNA target polynucleotides can be carried out in a multiplex fashion using principles of conventional polymerase chain reactions (PCR) and reverse-transcription polymerase chain reactions (RT-PCR), respectively.

In addition, the individual primer concentrations do not need to be optimized; using all primers at approximately equimolar concentrations can yield good results. The use of low primer concentrations reduces the possibility of non-specific primer interactions. Multiplex amplification of virtually any combination of sequences can be rapidly achieved without time-consuming optimization steps.

Through multiplexing, multiplex amplification reactions can be carried out using off-the-shelf commercially available reagents. Off-the-shelf reagents comprising amplification primers and probes can be pooled together and used in a multiplex amplification reaction without prior removal of probes. Like multiplex amplifications carried out in the absence of such oligonucleotide probes, multiplex amplifications carried out in the presence of such oligonucleotide probes can be divided into aliquots, with or without prior dilution, for subsequent analysis without further purification or manipulation.

The single-plex or multiplex amplification methods can be carried out in the presence of one or more intercalating dyes capable of producing a detectable signal upon binding to a double-stranded polynucleotide (e.g., SYBR® Green I or II, SYBR® Gold, ethidium bromide, or YO-PRO-1; Molecular Probes, Eugene, Oreg.).

Any suitable primer pair can be used, provided the pair has respective sequences designed to anneal to respective opposite ends of a sequence or segment of DNA desired to be replicated. For example, it might be desired to amplify and detect DNA of a known biofouling agent that is known to cause a bioburden in an industrial substance. The sequence or segment to be replicated and amplified can be a known or predetermined sequence of DNA that is found in (1) a specific organism, (2) a specific gene, (3) a type of organism such as a species, genus, family, order, class, phylum, kingdom, or domain of organism, or (4) a gene found in a specific type of organism.

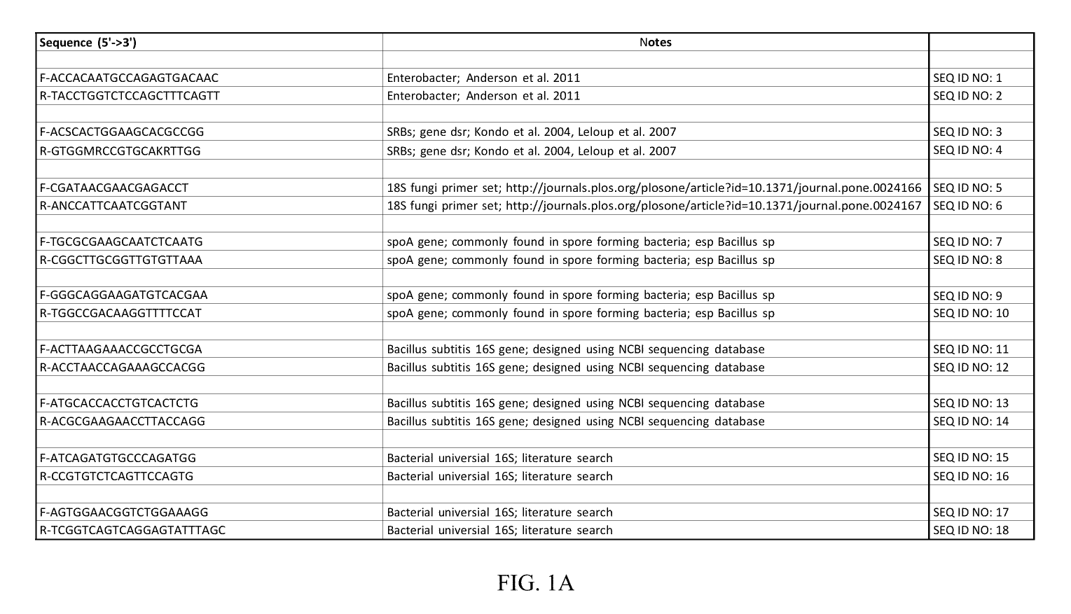

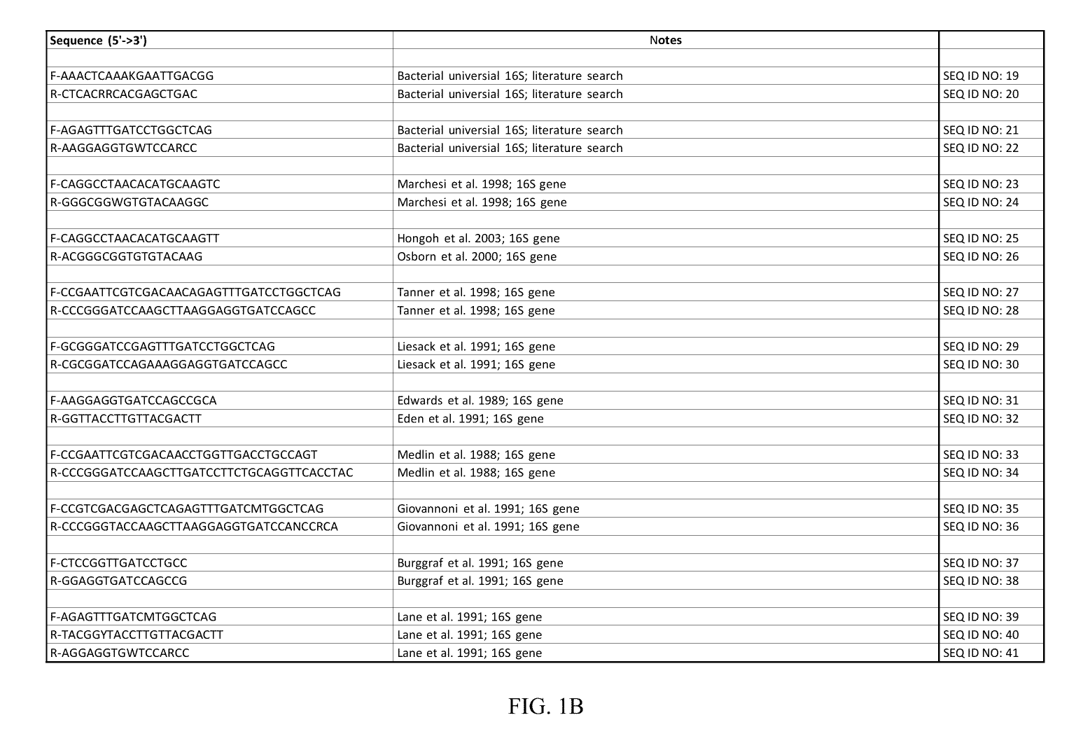

With reference to the figures, FIGS. 1A and 1B are parts one and two of a two-part table showing primers that can be used as primer pairs for replicating and amplifying DNA sequences of known biofouling organisms found in industrial substances. FIGS. 1A and 1B also show, in the “notes” column, the corresponding sources of literature wherein such primers are described and identified, and wherein respective sequences or genes targeted by the respective primers, are identified. The listed primers are grouped, almost entirely, as pairs, with each pair including a Forward primer designated “F-” and a Reverse primer designated “R-”. The “F-” and “R-” labels are not nucleotides but rather simply indicate whether the sequence following the hyphen (-) is the Forward primer or the Reverse primer of the pair. The last group, shown in FIG. 1B, is a set of three primers, wherein either of the two Reverse primers can be paired with the single Forward primer; different lengths of sequences, both starting at the same end at one end, can be tested for and enumerated depending on which Reverse primer is used.

A suitable pair of primers can be selected, ordered, and used to replicate the respective DNA segment of the corresponding organism or gene of interest. The first two entries can be used to make up a pair of primers for replicating a bacterial sequence of the DNA from the genus Enterobacter. For example, the first-listed primer can act as a forward primer and be used in conjunction with the second-listed primer that can act as a reverse primer, and the pair or set can be used to replicate and amplify a target sequence. In general, two consecutive primers on the list, identified from the same reference, can be used as a pair of primers that can be used together, including, for example, the first pair, second pair, and third pair listed in the table. For the many primers listed that can be used for replicating templates from the 16S gene, occurrences of two consecutive primers on the list, that are identified from the same reference, can be used as a pair of primers that together replicate a template from the 16S gene. The two primers listed from Tanner et al. represent a primer pair, the two primers listed from Liesack et al. represent a primer pair, the two primers listed from Medlin et al. represent a primer pair, the two primers listed from Giovannoni et al. represent a primer pair, and the two primers listed from Burggraf et al. represent a primer pair. Many combinations of two primers from the list of primers for replicating portions of the same gene, for example, the 16S gene, can be used together as primer pairs provided they polymerize in the correct orientation with respect to the double-stranded template.

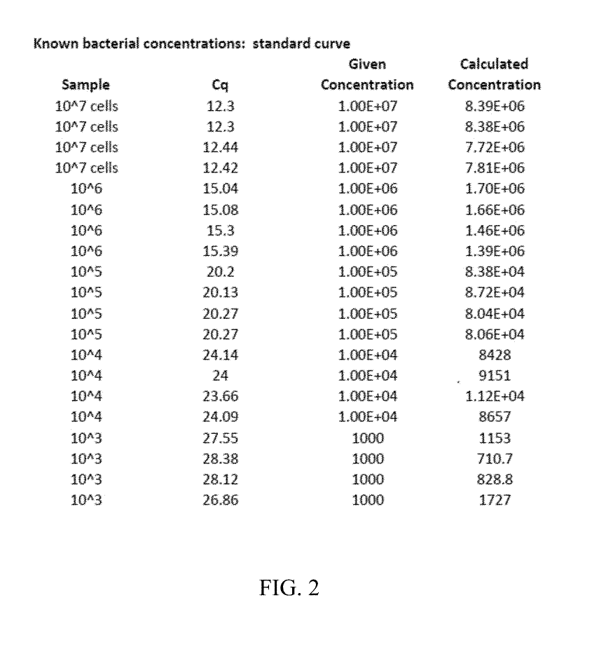

FIG. 2 is a table showing standard values of Cq of industrial samples having known biofouling agent cell concentrations per microliter (μL) and showing that calculated concentrations based on Cq measurements show good correspondence to the known, actual, given concentrations. To be used as a measure of quantity, the Cq of a sample can either be related to the Cq of another sample (often named a calibrator), to determine relative quantification, or be related to the Cq of a set of known copy number standards, to determine absolute quantification.

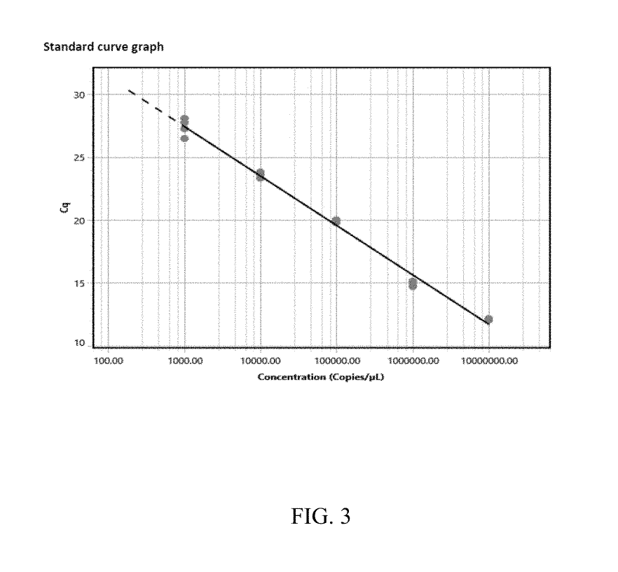

FIG. 3 is a graph showing a standard curve or line correlating the quantification cycle values (Cq) of the various serial dilutions of cells shown in FIG. 2. The cells are of a known biofouling organism. The data and/or graph can be used as a standard or set of standards, or to prepare a standard or set of standards, for example, to compare on-site test results of a sample from an industrial substance. Standards based on Cq or the cycle threshold value (Ct) can be used as, or used to generate, standards. The standards can be generated based on samples of known concentrations of known biofouling agents or organisms. The present methods can involve first preparing a standard or a set of standards with which on-site test results can be compared. Libraries of standards and libraries of sets of standards can be generated, stored, used, displayed, printed, kept, maintained, and updated.

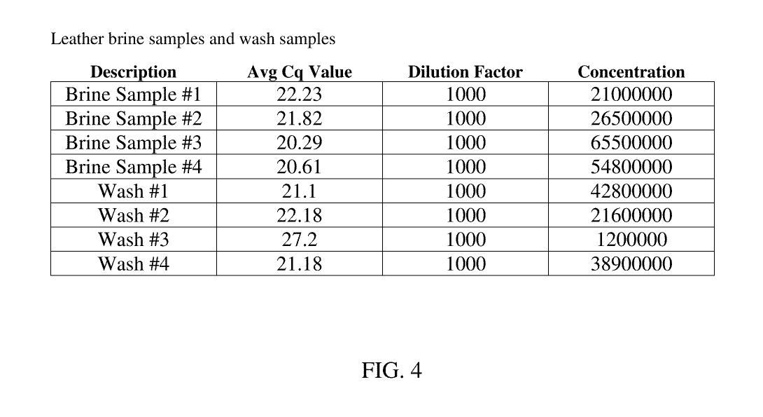

FIG. 4 is a table showing leather brine samples and wash samples that include biofouling agents that, when subject to PCR, generate Cq values indicating the concentrations of agents shown in the table. Each sample was diluted by a factor of 1000 before running the PCR used to generate the Cq values. Brine samples and respective wash samples were tested. The Wash #1 sample was generated by washing leather that had been tanned in Brine sample #1, the Wash #2 sample was generated by washing leather that had been tanned in Brine sample #2, and so on. The primer pair used to replicate and amplify bacteria present in the samples is shown as the first pair of primers in FIG. 1A, that is, the pair of primers for Enterobacter, described by Anderson et al. PCR was carried out using a MIC qPCR Cycler from Bio Molecular Systems, Taunton, Mass. The average Cq values were computed using the MIC software, also available from Bio Molecular Systems. Based on the concentrations determined, an effective amount of biocide can be dosed into the leather-treating brine without using or wasting excess biocide, making the leather-treating process less expensive and safer.

With the methods of the present invention, it is more possible to accurately and effectively control biological treatment reactors, guide biocide dosing programs, determine drinking water cleanliness, manage fermentation processes, assess soil activity, determine corrosion/deposit process types, measure equipment or products for microorganism contamination and the like.

The present invention includes the following aspects/embodiments/features in any order and/or in any combination:

1. The present invention relates to a method to quantify bioburden present in a substance, said method comprising:
a) obtaining a sample of the substance;
b) optionally filtering said sample;
c) optionally extracting DNA from the sample;
d) optionally diluting said sample;
e) conducting a polymerase chain reaction (PCR) with said sample or portion thereof utilizing a primer pair designed to target and amplify a segment of DNA of at least one organism that causes bioburden in said substance, and forming amplification data; and
f) correlating the amplification data with a standard, to determine an estimated amount or concentration of said organism in said substance,

wherein said substance is from, or supplied to, a papermaking plant, a pulp making plant, a leather making or processing plant, a fermentation facility, an oil and gas recovery site or production facility, a water treatment plant, a water cooling tower, or a sewage treatment plant.

2. The method of any preceding or following embodiment/feature/aspect, wherein said sample comprises endospores.
3. The method of any preceding or following embodiment/feature/aspect, wherein said sample comprises live organisms, viable cells from an organism, or a combination thereof.
4. The method of any preceding or following embodiment/feature/aspect, wherein said sample comprises endospores and viable cells, and said primer pair comprises at least two primer pairs, each primer pair targeting and amplifying a respective segment of DNA of at least one organism that causes bioburden in said substance.
5. The method of any preceding or following embodiment/feature/aspect, wherein said sample comprises endospores and viable cells, and said method further comprises isolating said endospores from said sample prior to conducting the PCR.
6. The method of any preceding or following embodiment/feature/aspect, wherein said sample comprises endospores and viable cells, and said method further comprises isolating said viable cells from said sample prior to conducting the PCR.
7. The method of any preceding or following embodiment/feature/aspect, wherein said substance primarily comprises a liquid.
8. The method of any preceding or following embodiment/feature/aspect, wherein said substance primarily comprises a solid.
9. The method of any preceding or following embodiment/feature/aspect, wherein said substance comprises a liquid and a solid, and said method further comprises substantially removing said solid before said step (e).
10. The method of any preceding or following embodiment/feature/aspect, further comprising treating said substance at a location having said substance, with at least one biocide or microbiocide, based on said estimated amount or concentration of said organism in said substance.
11. The method of any preceding or following embodiment/feature/aspect, further comprising treating said substance at a processing facility having, and configured to process, said substance, with at least one biocide or microbiocide administered at a dosage calculated based on said estimated amount or concentration of said organism in said substance.
12. The method of any preceding or following embodiment/feature/aspect, wherein the substance comprises viable cells of a biofouling organism and the obtaining a sample comprises lysing the viable cells.
13. The method of any preceding or following embodiment/feature/aspect, further comprising diluting the sample to form at least a first sample portion and a second sample portion, wherein the conducting a polymerase chain reaction comprises:

conducting a polymerase chain reaction (PCR) with said first sample portion, utilizing a primer pair to target and amplify a segment of DNA of at least one organism that is known to cause a bioburden in said substance;

conducting a polymerase chain reaction (PCR) with said second sample portion utilizing the primer pair;

correlating amplification data from amplification of the segment of DNA, determined from the PCR carried out on the first sample portion, with a standard or set of standards to determine a first estimated amount or concentration of said organism in said substance;

correlating amplification data from amplification of the segment of DNA, determined from the PCR carried out on the second sample portion, with the standard or set of standards to determine a second estimated amount or concentration of said organism in said substance;

comparing the first estimated amount or concentration with the second estimated amount or concentration and determining the average amount or concentration thereof; and

administering at least one biocide or microbiocide, to the substance, at a dosage calculated based on said average amount or concentration.

14. The method of any preceding or following embodiment/feature/aspect, wherein the PCR is conducted in the presence of a fluorescent resonance energy transfer (FRET) oligonucleotide probe, an extension phase of the PCR releases an unquenched reporter dye from the probe, and the method further comprises:

irradiating an excitation wavelength toward the sample, which excites the unquenched reporter dye, during each thermal cycle of the PCR; and

sensing the intensity of fluorescent emission during each thermal cycle of the PCR,

wherein the correlating a rate of amplification of the segment of DNA comprises graphing a curve of the intensity of fluorescent emission detected, per thermal cycle, to determine a quantitation cycle (Cq), and correlating the determined Cq to Cq values of known standards having known concentrations of the at least one organism.

15. The method of any preceding or following embodiment/feature/aspect, wherein primer pair comprises a primer pair designed to amplify a segment of a gene that appears in more than one different organism that is known to cause bioburden in said substance.
16. The method of any preceding or following embodiment/feature/aspect, wherein the primer pair comprises a primer pair designed to amplify one or more segments of DNA from bacteria.
17. The method of any preceding or following embodiment/feature/aspect, wherein primer pair comprises a primer pair designed to amplify one or more segments of DNA from one or more fungi.
18. The method of any preceding or following embodiment/feature/aspect, further comprising generating and detecting a signal that (1) is indicative of the presence of the segment of DNA, and (2) intensifies with an increasing concentration of amplicons of the segment of DNA, resulting from the PCR.
19. The method of any preceding or following embodiment/feature/aspect, further comprising preparing a set of standards, the set comprising results that have been plotted, graphed, saved, or tabulated, or any combination thereof, the preparing comprising conducting a polymerase chain reaction (PCR) on known samples that each comprise a known concentration of a known biofouling agent, wherein the PCR on each known sample uses the primer pair and amplifies the segment of DNA of the at least one organism, and the known samples comprise at least two known samples of different concentrations of the at least one organism.
20. The method of any preceding or following embodiment/feature/aspect, wherein the amplification data comprises a rate of amplification, a Cq value, a Ct value, or a combination thereof.
21. The method of any preceding or following embodiment/feature/aspect, wherein said method is performed at said papermaking plant, pulp making plant, leather making or processing plant, fermentation facility, oil and gas recovery site or production facility, water treatment plant, water cooling tower, or sewage treatment plant.
22. The method of any preceding or following embodiment/feature/aspect, wherein said method is conducted and results provided within 3 hours of said obtaining of said sample.
23. The method of any preceding or following embodiment/feature/aspect, wherein said method is performed in the absence of any DNA extraction step.
24. The method of any preceding or following embodiment/feature/aspect, wherein said method further comprises a DNA extraction step.

The present invention can include any combination of these various features or embodiments above and/or below as set forth in sentences and/or paragraphs. Any combination of disclosed features herein is considered part of the present invention and no limitation is intended with respect to combinable features.

Applicant specifically incorporates the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

What is claimed is:

1. A method to quantify bioburden present in a substance, said method comprising:
a) obtaining a sample of the substance;
b) optionally filtering said sample;
c) optionally extracting DNA from the sample;
d) optionally diluting said sample;
e) conducting a polymerase chain reaction (PCR) with said sample or portion thereof utilizing a primer pair designed to target and amplify a segment of DNA of at least one organism that causes bioburden in said substance, and forming amplification data; and
f) correlating the amplification data with a standard, to determine an estimated amount or concentration of said organism in said substance,
wherein said substance is from, or supplied to, a papermaking plant, a pulp making plant, a leather making or processing plant, a fermentation facility, an oil and gas recovery site or production facility, a water treatment plant, a water cooling tower, or a sewage treatment plant.

2. The method of claim 1, wherein said sample comprises endospores.

3. The method of claim 1, wherein said sample comprises live organisms, viable cells from an organism, or a combination thereof.

4. The method of claim 1, wherein said sample comprises endospores and viable cells, and said primer pair comprises at least two primer pairs, each primer pair targeting and amplifying a respective segment of DNA of at least one organism that causes bioburden in said substance.

5. The method of claim 1, wherein said sample comprises endospores and viable cells, and said method further comprises isolating said endospores from said sample prior to conducting the PCR.

6. The method of claim 1, wherein said sample comprises endospores and viable cells, and said method further comprises isolating said viable cells from said sample prior to conducting the PCR.

7. The method of claim 1, wherein said substance primarily comprises a liquid.

8. The method of claim 1, wherein said substance primarily comprises a solid.

9. The method of claim 1, wherein said substance comprises a liquid and a solid, and said method further comprises substantially removing said solid before said step (e).

10. The method of claim 1, further comprising treating said substance at a location having said substance, with at least one biocide or microbiocide, based on said estimated amount or concentration of said organism in said substance.

11. The method of claim 1, further comprising treating said substance at a processing facility having, and configured to process, said substance, with at least one biocide or microbiocide administered at a dosage calculated based on said estimated amount or concentration of said organism in said substance.

12. The method of claim 1, wherein the substance comprises viable cells of a biofouling organism and the obtaining a sample comprises lysing the viable cells.

13. The method of claim 1, further comprising diluting the sample to form at least a first sample portion and a second sample portion, wherein the conducting a polymerase chain reaction comprises:
conducting a polymerase chain reaction (PCR) with said first sample portion, utilizing a primer pair to target and amplify a segment of DNA of at least one organism that is known to cause a bioburden in said substance;
conducting a polymerase chain reaction (PCR) with said second sample portion utilizing the primer pair;
correlating amplification data from amplification of the segment of DNA, determined from the PCR carried out on the first sample portion, with a standard or set of standards to determine a first estimated amount or concentration of said organism in said substance;
correlating amplification data from amplification of the segment of DNA, determined from the PCR carried out on the second sample portion, with the standard or set of standards to determine a second estimated amount or concentration of said organism in said substance;
comparing the first estimated amount or concentration with the second estimated amount or concentration and determining the average amount or concentration thereof; and
administering at least one biocide or microbiocide, to the substance, at a dosage calculated based on said average amount or concentration.

14. The method of claim 1, wherein the PCR is conducted in the presence of a fluorescent resonance energy transfer (FRET) oligonucleotide probe, an extension phase of the PCR releases an unquenched reporter dye from the probe, and the method further comprises:
irradiating an excitation wavelength toward the sample, which excites the unquenched reporter dye, during each thermal cycle of the PCR; and
sensing the intensity of fluorescent emission during each thermal cycle of the PCR,
wherein the correlating a rate of amplification of the segment of DNA comprises graphing a curve of the intensity of fluorescent emission detected, per thermal cycle, to determine a quantitation cycle (Cq), and correlating the determined Cq to Cq values of known standards having known concentrations of the at least one organism.

15. The method of claim 1, wherein primer pair comprises a primer pair designed to amplify a segment of a gene that appears in more than one different organism that is known to cause bioburden in said substance.

16. The method of claim 1, wherein the primer pair comprises a primer pair designed to amplify one or more segments of DNA from bacteria.

17. The method of claim 1, wherein primer pair comprises a primer pair designed to amplify one or more segments of DNA from one or more fungi.

18. The method of claim 1, further comprising generating and detecting a signal that (1) is indicative of the presence of the segment of DNA, and (2) intensifies with an increasing concentration of amplicons of the segment of DNA, resulting from the PCR.

19. The method of claim 1, further comprising preparing a set of standards, the set comprising results that have been plotted, graphed, saved, or tabulated, or any combination thereof, the preparing comprising conducting a polymerase chain reaction (PCR) on known samples that each comprise a known concentration of a known biofouling agent, wherein the PCR on each known sample uses the primer pair and amplifies the segment of DNA of the at least one organism, and the known samples comprise at least two known samples of different concentrations of the at least one organism.

20. The method of claim 1, wherein the amplification data comprises a rate of amplification, a Cq value, a Ct value, or a combination thereof.

21. The method of claim 1, wherein said method is performed at said papermaking plant, pulp making plant, leather making or processing plant, fermentation facility, oil and gas recovery site or production facility, water treatment plant, water cooling tower, or sewage treatment plant.

22. The method of claim 1, wherein said method is conducted and results provided within 3 hours of said obtaining of said sample.

23. The method of claim 1, wherein said method is performed in the absence of any DNA extraction step.

24. The method of claim 1, wherein said method further comprises a DNA extraction step.


SEQUENCE LISTING (41 sequences)

SEQ ID NO: 1 - DNA, 22 bp, unknown
  DNA primer derived from a bacterium from genus Entobacter described in Anderson et al. (2011).
accacaatgccagagtgacaac

SEQ ID NO: 2 - DNA, 23 bp, unknown
  DNA primer derived from a bacterium from genus Entobacter described in Anderson et al. (2011).
tacctggtctccagctttcagtt

SEQ ID NO: 3 - DNA, 20 bp, unknown
  DNA primer derived from a bacterium from genus SRBs, gene dsr, described in Kondo et al. (2004) and Leloup et al. (2007).
acscactggaagcacgccgg

SEQ ID NO: 4 - DNA, 19 bp, unknown
  DNA primer derived from a bacterium from genus SRBs, gene dsr, described in Kondo et al. (2004) and Leloup et al. (2007).
gtggmrccgtgcakrttgg

SEQ ID NO: 5 - DNA, 18 bp, unknown
  DNA primer derived from 18S Fungi primer set, described at http//journals.plos.org/plosone/article?id=10.1371/ journal.pone.0024166
cgataacgaacgagacct

SEQ ID NO: 6 - DNA, 18 bp, unknown
  DNA primer derived from 18S Fungi primer set, described at http//journals.plos.org/plosone/article?id=10.1371/ journal.pone.0024166
  misc_feature (2)..(2): n is a, c, g, or t
  misc_feature (17)..(17): n is a, c, g, or t
anccattcaatcggtant

SEQ ID NO: 7 - DNA, 20 bp, unknown
  DNA primer of a spoA gene, derived from a spore forming bacterium from genus Bacillus
tgcgcgaagcaatctcaatg

SEQ ID NO: 8 - DNA, 20 bp, unknown
  DNA primer of a spoA gene, derived from a spore forming bacterium from genus Bacillus
cggcttgcggttgtgttaaa

SEQ ID NO: 9 - DNA, 20 bp, unknown
  DNA primer of a spoA gene, derived from a spore forming bacterium from genus Bacillus
gggcaggaagatgtcacgaa

SEQ ID NO: 10 - DNA, 20 bp, unknown
  A spoA gene, derived from a spore forming bacterium from genus Bacillus
tggccgacaaggttttccat

SEQ ID NO: 11 - DNA, 20 bp, unknown
  A Bacillus subtitis 16S gene, designed using NCBI sequencing database
acttaagaaaccgcctgcga

SEQ ID NO: 12 - DNA, 20 bp, unknown
  A Bacillus subtitis 16S gene, designed using NCBI sequencing database
acctaaccagaaagccacgg

SEQ ID NO: 13 - DNA, 20 bp, unknown
  A Bacillus subtitis 16S gene, designed using NCBI sequencing database
atgcaccacctgtcactctg

SEQ ID NO: 14 - DNA, 20 bp, unknown
  A Bacillus subtitis 16S gene, designed using NCBI sequencing database
acgcgaagaaccttaccagg

SEQ ID NO: 15 - DNA, 19 bp, unknown
  A Bacillus universial 16S gene, found via literature search
atcagatgtgcccagatgg

SEQ ID NO: 16 - DNA, 19 bp, unknown
  A Bacillus universial 16S gene, found via literature search
ccgtgtctcagttccagtg

SEQ ID NO: 17 - DNA, 20 bp, unknown
  A Bacillus universial 16S gene, found via literature search
agtggaacggtctggaaagg

SEQ ID NO: 18 - DNA, 23 bp, unknown
  A Bacillus universial 16S gene, found via literature search
tcggtcagtcaggagtatttagc

SEQ ID NO: 19 - DNA, 20 bp, unknown
  A Bacillus universial 16S gene, found via literature search
aaactcaaakgaattgacgg

SEQ ID NO: 20 - DNA, 18 bp, unknown
  A Bacillus universial 16S gene, found via literature search
ctcacrrcacgagctgac

SEQ ID NO: 21 - DNA, 20 bp, unknown
  A Bacillus universial 16S gene, found via literature search
agagtttgatcctggctcag

SEQ ID NO: 22 - DNA, 17 bp, unknown
  A Bacillus universial 16S gene, found via literature search
aaggaggtgwtccarcc

SEQ ID NO: 23 - DNA, 21 bp, unknown
  A 16S gene described in Marchesi et al. (1998)
caggcctaacacatgcaagtc

SEQ ID NO: 24 - DNA, 18 bp, unknown
  A 16S gene described in Marchesi et al. (1998)
gggcggwgtgtacaaggc

SEQ ID NO: 25 - DNA, 21 bp, unknown
  A 16S gene described in Hongoh et al. (2003)
caggcctaacacatgcaagtt

SEQ ID NO: 26 - DNA, 18 bp, unknown
  A 16S gene described in Osborn et al. (2000)
acgggcggtgtgtacaag

SEQ ID NO: 27 - DNA, 37 bp, unknown
  A 16S gene described in Tanner et al. (1998)
ccgaattcgtcgacaacagagtttgatcctggctcag

SEQ ID NO: 28 - DNA, 33 bp, unknown
  A 16S gene described in Tanner et al. (1998)
cccgggatccaagcttaaggaggtgatccagcc

SEQ ID NO: 29 - DNA, 28 bp, unknown
  A 16S gene described in Liesack et al. (1991)
gcgggatccgagtttgatcctggctcag

SEQ ID NO: 30 - DNA, 29 bp, unknown
  A 16S gene described in Liesack et al. (1991)
cgcggatccagaaaggaggtgatccagcc

SEQ ID NO: 31 - DNA, 20 bp, unknown
  A 16S gene described in Edwards et al. (1989)
aaggaggtgatccagccgca

SEQ ID NO: 32 - DNA, 19 bp, unknown
  A 16S gene described in Eden et al. (1991)
ggttaccttgttacgactt

SEQ ID NO: 33 - DNA, 34 bp, unknown
  A 16S gene described in Medlin et al. (1988)
ccgaattcgtcgacaacctggttgacctgccagt

SEQ ID NO: 34 - DNA, 39 bp, unknown
  A 16S gene described in Medlin et al. (1988)
cccgggatccaagcttgatccttctgcaggttcacctac

SEQ ID NO: 35 - DNA, 34 bp, unknown
  A 16S gene described in Giovannoni et al. (1991)
ccgtcgacgagctcagagtttgatcmtggctcag

SEQ ID NO: 36 - DNA, 36 bp, unknown
  A 16S gene described in Giovannoni et al. (1991)
  misc_feature (31)..(31): n is a, c, g, or t
cccgggtaccaagcttaaggaggtgatccanccrca

SEQ ID NO: 37 - DNA, 17 bp, unknown
  A 16S gene described in Burggraf et al. (1991)
ctccggttgatcctgcc

SEQ ID NO: 38 - DNA, 16 bp, unknown
  A 16S gene described in Burggraf et al. (1991)
ggaggtgatccagccg

SEQ ID NO: 39 - DNA, 20 bp, unknown
  A 16S gene described in Lane et al. (1991)
agagtttgatcmtggctcag

SEQ ID NO: 40 - DNA, 22 bp, unknown
  A 16S gene described in Lane et al. (1991)
tacggytaccttgttacgactt

SEQ ID NO: 41 - DNA, 16 bp, unknown
  A 16S gene described in Lane et al. (1991)
aggaggtgwtccarcc